(19) United States
(12) Patent Application Publication
(10) Pub. No.: US 2011/0305637 A1
(43) Pub. Date: Dec. 15, 2011

(54) RADIOLABELLED PDE10 LIGANDS

(75) Inventors: José Ignacio Andrés-Gil, Madrid (ES); Guy Maurits R. Bormans, Rotselaar (BE); Sofie Jeanne Leopoldine Celen, Tessenderlo (BE); Meri De Angelis, Toledo (ES)

(73) Assignee: Janssen Pharmaceutica N.V.

(21) Appl. No.: 13/203,026

(22) PCT Filed: Feb. 22, 2010
(86) PCT No.: PCT/EP2010/052214

§ 371 Date: Aug. 24, 2011

(30) Foreign Application Priority Data
Feb. 24, 2009 (EP) 09153524.5

(51) Int. Cl. A61K 51/04; C07D 401/14

(52) U.S. Cl. 424/1.89; 546/176

(57) ABSTRACT

The present invention relates to novel, selective, radiolabeled PDE10 ligands which are useful for imaging and quantifying the PDE10A enzyme in tissues using positron-emission tomography (PET). The invention is also directed to compositions comprising such compounds, to processes for preparing such compounds and compositions, and to the use of such compounds and compositions for imaging a tissue, cells or a host, in vitro or in vivo.

### FIELD OF THE INVENTION

The present invention relates to novel, selective, radiolabeled PDE10 ligands which are useful for imaging and quantifying the PDE10A enzyme in tissues, using positron-emission tomography (PET). The invention is also directed to compositions comprising such compounds, to processes for preparing such compounds and compositions, and to the use of such compounds and compositions for imaging a tissue, cells or a host, in vitro or in vivo.

### BACKGROUND OF THE INVENTION

Phosphodiesterases (PDEs) are a family of enzymes encoded by 21 genes and subdivided into 11 distinct families according to structural and functional properties. These enzymes metabolically inactivate widely occurring intracellular second messengers, 3′,5′-cyclic adenosine monophosphate (cAMP) and 3′,5′-cyclic guanosine monophosphate (cGMP). These two messengers regulate a wide variety of biological processes, including pro-inflammatory mediator production and action, ion channel function, muscle contraction, learning, differentiation, apoptosis, lipogenesis, glycogenolysis, and gluconeogenesis. They do this by activation of protein kinase A (PKA) and protein kinase G (PKG), which in turn phosphorylate a wide variety of substrates including transcription factors and ion channels that regulate innumerable physiological responses. In neurons, this includes the activation of cAMP and cGMP-dependent kinases and subsequent phosphorylation of proteins involved in acute regulation of synaptic transmission as well as in neuronal differentiation and survival. Intracellular concentrations of cAMP and cGMP are strictly regulated by the rate of biosynthesis by cyclases and by the rate of degradation by PDEs. PDEs are hydrolases that inactivate cAMP and cGMP by catalytic hydrolysis of the 3′-ester bond, forming the inactive 5′-monophosphate (Scheme 1).

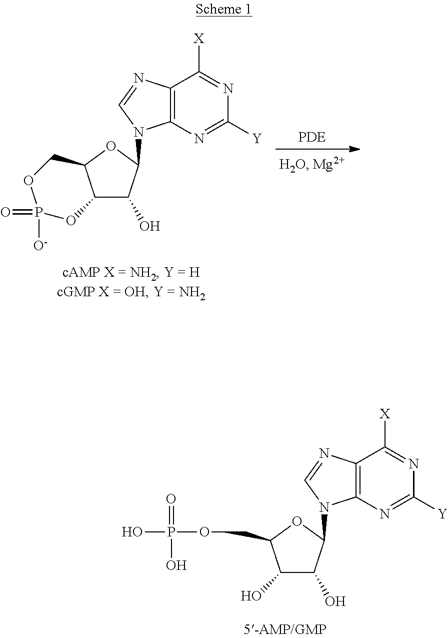

On the basis of substrate specificity, the PDE families can be divided into three groups: i) the cAMP-specific PDEs, which include PDE4, -7 and -8; ii) the cGMP-selective enzymes PDES and -9; and iii) the dual-substrate PDEs, PDE1, -2 and -3, as well as PDE10 and -11. The discovery of phosphodiesterase 10A (PDE10A) was reported in 1999. Of all the 11 known PDE families, PDE10A has most restricted distribution with high expression only in the brain and testes. In the brain, PDE10A mRNA and protein are highly expressed in the striatum. This unique distribution of PDE10A in the brain, together with its increased pharmacological characterization, indicates a potential use of PDE10A inhibitors for treating neurological and psychiatric disorders like schizophrenia. Our aim was to develop a positron emission tomography (PET) imaging agent to quantify the PDE10A enzyme in the brain. PET is a noninvasive imaging technique that offers highest spatial and temporal resolution of all nuclear imaging techniques and has the added advantage that it can allow for true quantification of tracer concentrations in tissues. It uses positron emitting radionuclides such as ¹⁵O, ¹³N, ¹¹C and ¹⁸F for detection.

WO-2006/072828 (Pfizer) discloses heteroaromatic quinoline compounds as selective PDE10 inhibitors.

### DETAILED DESCRIPTION OF THE INVENTION

The present invention relates to compounds having the Formula (I)

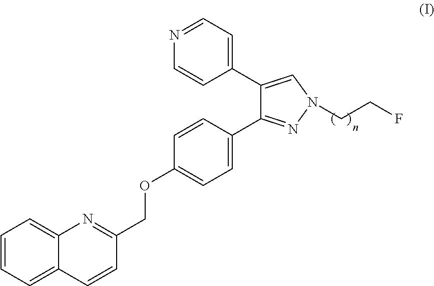

wherein n is 1 or 2, and F is [¹⁸F], and the acceptable salts and solvates thereof.

Acceptable salts of the compounds of formula (I) are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are included within the ambit of the present invention. The pharmaceutically acceptable salts are defined to comprise the therapeutically active non-toxic acid addition salt forms that the compounds according to Formula (I) are able to form. Said salts can be obtained by treating the base form of the compounds according to Formula (I) with appropriate acids, for example inorganic acids, for example hydrohalic acid, in particular hydrochloric acid, hydrobromic acid, sulphuric acid, nitric acid and phosphoric acid; organic acids, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzenesulfonic acid, p-toluenesulfonic acid, cyclamic acid, salicylic acid, p-aminosalicylic acid and pamoic acid.

Conversely said salt forms can be converted into the free base form by treatment with an appropriate base.

### Preparation

The compounds according to the invention can generally be prepared by a succession of steps, each of which is known to the skilled person. In particular, the compounds can be prepared according to the following synthesis methods.

Compounds of Formula (I) in their non-radiolabeled version can be prepared by synthesis methods well known by the person skilled in the art. Compounds of the invention may be prepared, for example, by the reaction sequence shown in Scheme 2. The synthesis of the starting material (II) is described in WO 2006/072828 (Pfizer).

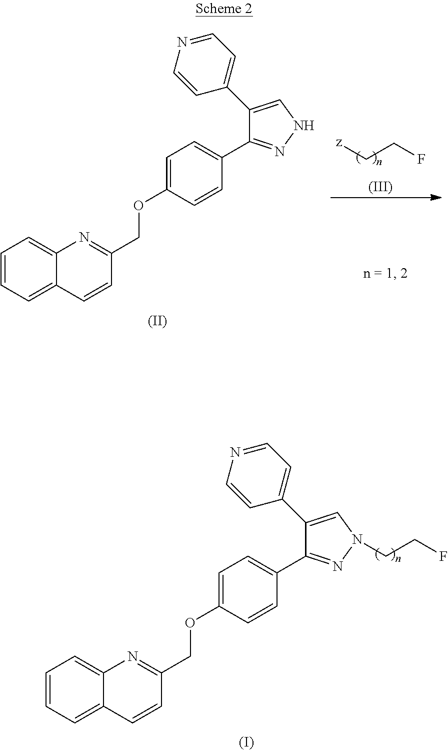

Therefore, a compound of Formula (II) may be reacted with a commercially available alkylating agent of Formula (III), in which Z is a suitable leaving group such as halo, for example bromo or iodo, in the presence of a suitable base such as cesium carbonate or potassium carbonate, in an inert solvent such as, for example, dimethylformamide, stirring the reaction mixture at a suitable temperature, typically at 100-150° C., using conventional heating or under microwave irradiation, for the required time to achieve completion of the reaction, typically 10 minutes in a microwave oven. The alkylation reaction usually affords a mixture of the two possible regioisomers, derived from the alkylation on both nitrogen atoms of the pyrazole ring, which can be separated by chromatographic methods, either by column chromatography or HPLC.

Alternatively, Z may be a hydroxyl group, in which case reaction with compound (II) can be performed using conventional Mitsunobu conditions, which are well known by the person skilled in the art. Thus, compound (II) can be reacted with compound (III) in which Z is hydroxyl- in the presence of diethyl- or diisopropyl azodicarboxylate and triphenylphosphine, in an inert solvent such as for example tetrahydrofuran, stirring the reaction mixture at a suitable temperature, typically at 100° C. under microwave irradiation, for a suitable period of time to allow completion of the reaction, typically 20 minutes. The Mitsunobu reaction usually affords a mixture of the two possible regioisomers, derived from the alkylation on both nitrogen atoms of the pyrazole ring, which can be separated by chromatographic methods, either by column chromatography or HPLC.

Alternatively, compounds of formula (I) can also be prepared by a reaction sequence as shown in scheme 3.

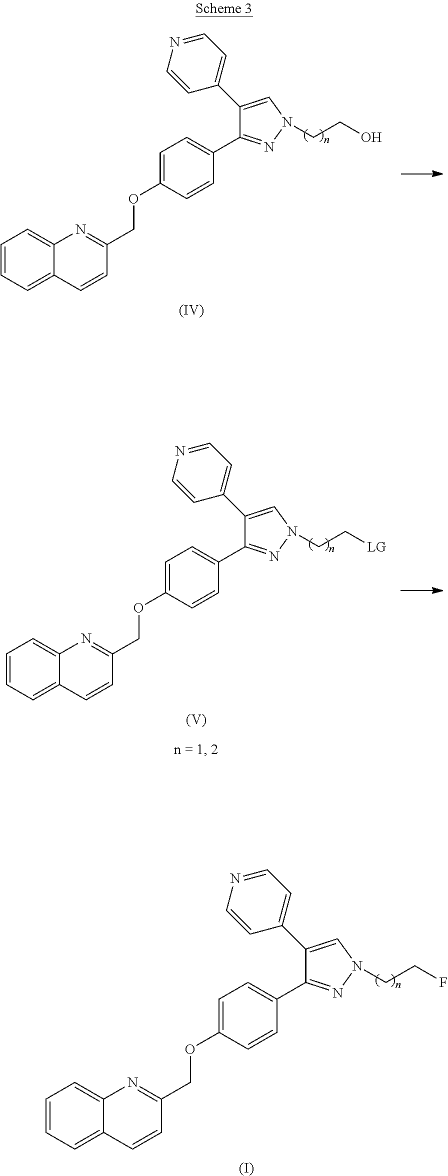

Therefore, in a compound of formula (IV) the hydroxyl group can be transformed into a suitable leaving group LG, such as methanesulfonate or tosylate, by methods well known by those skilled in the art, affording an intermediate of formula (V). Then the leaving group can be replaced by fluorine using standard methods well known by the skilled person, such as, for example, reacting with tetrabutylammonium fluoride in an inert solvent such as for example tetrahydrofuran, stirring the reaction mixture at a suitable temperature, typically at 70° C. under microwave irradiation, for a suitable period of time to allow completion of the reaction, typically 10 minutes. Alternatively, compounds of formula (I) can also be prepared by direct reaction of an intermediate of formula (IV) with a fluorinating agent such as, for example, (N,N-diethylamino)sulphur trifluoride (DAST), by art known procedures.

The incorporation of radioactive fluorine atoms into the compounds of formula (I) may be performed using techniques known in the art, for example, by reaction of a suitable precursor of formula (V) with a nucleophilic radioactive fluorinating reagent, such as K[¹⁸F]/Kryptofix® 222 or tetraalkyl ammonium salts incorporating radioactive fluoride, in an inert solvent such as, for example, dimethylformamide, stirring the reaction mixture at a suitable temperature, typically at 100° C., using conventional heating or under microwave irradiation, for the required time to achieve completion of the reaction, typically 10 minutes in a microwave oven.

The radiosynthesis of [¹⁸F]I-b wherein n=2 was carried out using the O-mesyl derivative of (IV-b) as a precursor and [¹⁸F]fluoride as a labeling agent. The reaction was carried out in anhydrous DMF with microwave assisted heating at 120° C. for 60 seconds by using 35 W power. The radiolabeled product was purified using a semi-preparative HPLC system and formulated using saline after sterile filtration. The radiochemical purity of this formulation as analyzed on an analytical HPLC system was found to be >99%. [¹⁸F]I-b was obtained in moderate radiochemical yields (15-27%). The identity of [¹⁸F]I-b was confirmed by co-elution with its non-radioactive analogue (I-b) after co-injection on to the same analytical HPLC system. The specific radioactivity of the tracer was found to be between 470 and 630 GBq/μmol.

The radiosynthesis of [¹⁸F]I-a wherein n=1 was carried out using the O-mesyl derivative (IV-a) as a precursor and [¹⁸F]fluoride as a labeling agent. The reaction was carried out in anhydrous DMF with conventional heating at 90° C. for 10 min. The radiolabeled product was purified using semi-preparative HPLC system and formulated using saline after sterile filtration. The radiochemical purity of this formulation as analyzed on an analytical HPLC system was found to be >99%. [¹⁸F]I-a was obtained in low to moderate radiochemical yields (9-14%). The identity of [¹⁸F]I-a was confirmed by co-elution with its non-radioactive analogue (I-a) after co-injection on to the same analytical HPLC system.

Intermediate compounds of formula (IV) can be prepared by synthesis methods well known by the person skilled in the art, such as, for example, by the reaction sequence shown in Scheme 4.

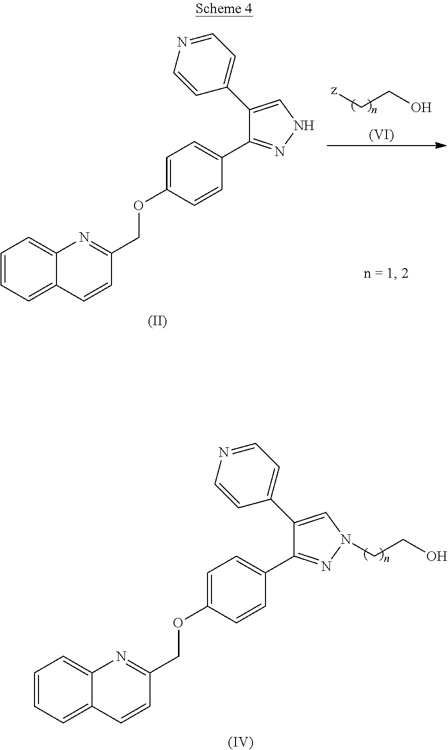

Therefore, compound of Formula (II) may be reacted with a commercially available alkylating agent of Formula (VI), in which Z is a suitable leaving group such as halo, bromo being the most preferred, in the presence of a suitable base such as cesium carbonate or potassium carbonate, in an inert solvent such as, for example, dimethylformamide, stirring the reaction mixture at a suitable temperature, typically at 100° C., using conventional heating or under microwave irradiation, for the required time to achieve completion of the reaction, typically 10 minutes in a microwave oven. The alkylation reaction usually affords a mixture of the two possible regioisomers, derived from the alkylation on both nitrogen atoms of the pyrazole ring, which can be separated by chromatographic methods, either by column chromatography or HPLC. Alternatively, compounds of formula (IV-a) where n=1 can also be prepared by the sequence of reactions shown in scheme 5.

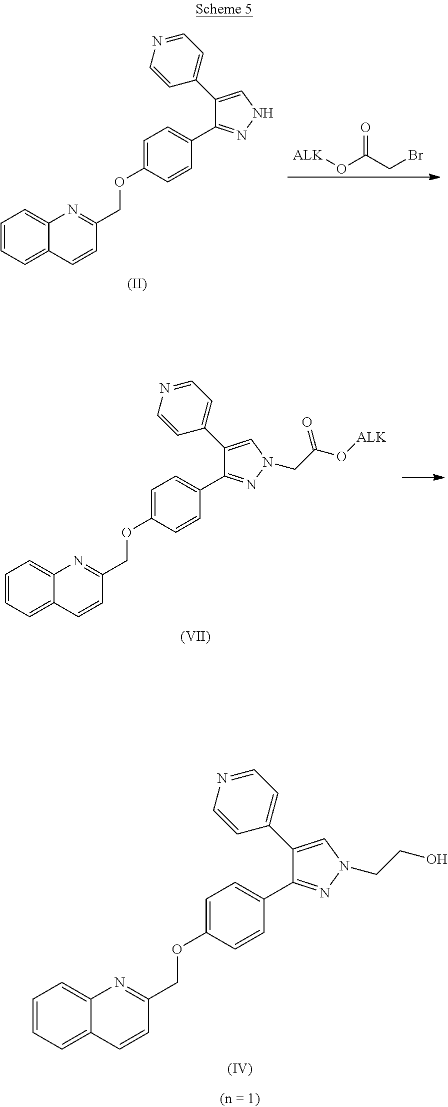

Thus, compound of Formula (II) may be reacted with commercially available methyl- or ethyl bromoacetate, in the presence of a suitable base such as cesium carbonate or potassium carbonate, in an inert solvent such as, for example, dimethylformamide, stirring the reaction mixture at a suitable temperature, typically at room temperature, for the required time to achieve completion of the reaction, typically 3 hours, to yield compound of formula (VII). The ester group can be reduced to alcohol by synthesis methods well known by the person skilled in the art, such as, for example, reaction with sodium borohydride or sodium cyanoborohydride, in a suitable inert solvent or mixture of solvents, such as for example dichloromethane and methanol, stirring the reaction mixture at a suitable temperature, typically at room temperature, for the required time to achieve completion of the reaction, typically 2 hours, to afford the compound of formula (IV-a) where n=1. The alkylation reaction usually affords a mixture of the two possible regioisomers, derived from the alkylation on both nitrogen atoms of the pyrazole ring, which can be separated by chromatographic methods, either by column chromatography or HPLC.

The affinity of compounds Ia and Ib was determined by measuring rat PDE10A2-cAMP inhibition:

Ia pIC50=9.32, selective over other PDEs>1000 fold
Ib pIC50=9.16, selective over other PDEs>1000 fold, except for PDE8A1>200 fold.

### Applications

The compounds according to the present invention find various applications for imaging tissues, cells or a host, both in vitro and in vivo. Thus, for instance, they can be used to map the differential distribution of PDE10 in subjects of different age and sex. Further, they allow one to explore for differential distribution of PDE10 in subjects afflicted by different diseases or disorders. Thus, abnormal distribution may be helpful in diagnosis, case finding, stratification of subject populations, and in monitoring disease progression in individual subjects. The radioligand may further find utility in determining PDE10A site occupancy by other ligands.

### Experimental Part

### Chemistry:

Hereinafter, the term “LCMS” means liquid chromatography/mass spectrometry, “GCMS” means gas chromatography/mass spectrometry, “HPLC” means high-performance liquid chromatography, “DCM” means dichloromethane, “DMF” means dimethylformamide, “EtOAc” means ethyl acetate, “THF” means tetrahydrofuran, “min.” means minutes, “h.” means hours, “Rt” means retention time (in minutes), “[M+H]⁺” means the protonated mass of the free base of the compound, “[M−H]⁻” means the deprotonated mass of the free base of the compound, ‘m.p.” means melting point.

Microwave assisted reactions were performed in a single-mode reactor: Emrys™ Optimizer microwave reactor (Personal Chemistry A.B., currently Biotage).

Thin layer chromatography (TLC) was carried out on silica gel 60 F254 plates (Merck) using reagent grade solvents. Flash column chromatography was performed on silica gel, particle size 60 Å, mesh=230-400 (Merck) under standard techniques. Automated flash column chromatography was performed using ready-to-connect cartridges from Merck, on irregular silica gel, particle size 15-40 μm (normal phase disposable flash columns) on an SPOT or FLASH system from Armen Instrument.

### A. Synthesis of Intermediates and Precursors

### Intermediate 1

### 3-{4-Pyridin-4-yl-3-[4-(quinolin-2-ylmethoxy)-phenyl]-pyrazol-1-yl}-propan-1-ol (I-1)

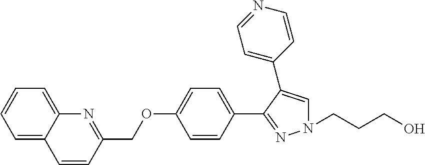

A mixture of intermediate 2-[4-(4-pyridin-4-yl-1H-pyrazol-3-yl)-phenoxymethyl]-quinoline (0.400 g, 1.057 mmol) that was synthesized following the method described in patent application WO 2006/072828, 3-bromopropan-1-ol (0.125 mL, 1.374 mmol) and cesium carbonate (1.033 g, 3.171 mmol) in DMF as solvent (5 mL) was heated in a microwave oven at 100° C. for 10 min. After cooling to room temperature the reaction mixture was quenched with water and extracted with EtOAc. The organic layer was separated, filtered over cotton and the solvents were evaporated in vacuo. The crude residue was purified by flash column chromatography (silicagel; EtOAc/methanol 100/0 to 90/10). The desired fractions were collected and evaporated in vacuo to yield the desired target compound I-1 (0.120 g, 26.0%). ¹H NMR (500 MHz, CDCl₃) δ ppm 2.09-2.17 (m, 2 H), 2.73 (br. s., 1H), 3.73 (t, J=5.8 Hz, 2 H), 4.34 (t, J=6.5 Hz, 2 H), 5.41 (s, 2 H), 6.99-7.04 (m, 2 H), 7.15-7.19 (m, 2 H), 7.36-7.41 (m, 2 H), 7.53-7.58 (m, 1H), 7.62 (s, 1H), 7.68 (d, J=8.7 Hz, 1H), 7.74 (ddd, J=8.4, 7.1, 1.3 Hz, 1H), 7.84 (d, J=8.1 Hz, 1H), 8.08 (d, J=8.4 Hz, 1H), 8.20 (d, J=8.4 Hz, 1H), 8.47 (br. d, J=5.8 Hz, 2 H). C₂₇H₂₄N₄O₂. LCMS: Rt 3.9, m/z 437 [M+H]⁺.

The corresponding regioisomer I-1′ 3-{4-Pyridin-4-yl-5-[4-(quinolin-2-ylmethoxy)-phenyl]-pyrazol-1-yl}-propan-1-ol was also isolated from the chromatographic purification (0.140 g, 24.3%). C₂₇H₂₄N₄O₂.

### Intermediate 2

### Methanesulfonic acid 3-{4-pyridin-4-yl-3-[4-(quinolin-2-ylmethoxy)-phenyl]-pyrazol-1-yl}-propyl ester (I-2)

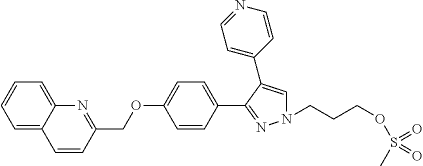

### a) Method 1

To a stirred solution of intermediate I-1 (0.170 g, 0.389 mmol) in DCM (3 mL) cooled at 0° C. were added pyridine (0.378 mL, 4.673 mmol) and methanesulfonyl chloride (0.242 mL, 3.116 mmol). The reaction mixture was stirred at 0° C. for 1 h. The mixture was quenched with water and extracted with more DCM. The organic solvent was separated, dried over sodium sulphate and evaporated to dryness in vacuo. The crude residue was purified by column chromatography (silicagel; EtOAc/methanol 100/0 to 95/5). The desired fractions were collected and evaporated in vacuo to yield I-2 (0.150 g, 75%) as a pale yellow oil. ¹H NMR (400 MHz, CDCl₃) δ ppm 2.35-2.43 (m, 2 H), 3.04 (s, 3 H), 4.29 (t, J=5.7 Hz, 2 H), 4.32 (t, J=6.5 Hz, 2 H), 5.41 (s, 2 H), 7.02 (d, J=8.8 Hz, 2 H), 7.16-7.20 (m, 2 H), 7.38 (d, J=8.8 Hz, 2 H), 7.51-7.59 (m, 1H), 7.68 (d, J=8.3 Hz, 1H), 7.68 (s, 1H), 7.71-7.77 (m, 1H), 7.83 (d, J=8.1 Hz, 1H), 8.08 (d, J=8.3 Hz, 1H), 8.20 (d, J=8.6 Hz, 1H), 8.45-8.50 (m, 2 H). C₂₈H₂₆N₄O₄S LCMS: Rt 3.1, m/z 515 [M+H]⁺ (elimination by-product is observed at m/z 419).

### b) Method 2

To a solution of I-1 (5 mg, 11.454 μmol) in dichloromethane (0.5 mL) was added pyridine (11 μL, 136.70 μmol) and this solution was stirred at 0° C. Methane sulfonyl chloride (7 μL, 90.44 μmol) was then added and the stirring was continued for an hour at 0° C. after which the solvent was evaporated by flushing with nitrogen. The crude mixture was redissolved in methanol (0.5 mL), diluted with water (4.5 mL) and passed through a C₁₈ SepPak® cartridge (Waters Instruments) that was preconditioned with methanol (3 mL) and milliQ® water (Millipore corp.) (6 mL). The cartridge was then rinsed with an additional volume of water (2 mL) to remove unreacted mesyl chloride. The product was eluted from the cartridge using acetonitrile (3 mL) and the solvents were evaporated under reduced pressure. Prior to the evaporation of solvents, HPLC analysis was performed to examine the conversion of precursor I-1 to its O-mesyl derivative. This HPLC analysis was done on an analytical XTerra™ RP C₁₈ column (Waters), which was eluted with gradient mixtures of water and acetonitrile (0 min: 95:5 v/v, 25 min: 10:90 v/v, 30 min: 10:90 v/v, linear gradient) at a flow rate of 1 mL/min. The analysis showed that the conversion rate was between 80 and 90%. Residual water was removed by azeotropic distillation with acetonitrile and the mixture was dried in the vacuum oven. On the day of radiolabeling experiments, which was usually the next morning, this reaction product was dissolved in anhydrous DMF (1.5 mL) and used (0.3 mL) for the direct nucleophilic radiofluorination reaction.

### Intermediate 3

### {4-Pyridin-4-yl-3-[4-(quinolin-2-ylmethoxy)-phenyl]-pyrazol-1-yl}-acetic acid methyl ester (I-3) and {4-Pyridin-4-yl-5-[4-(quinolin-2-ylmethoxy)-phenyl]-pyrazol-1-yl}-acetic acid methyl ester (I-3′)

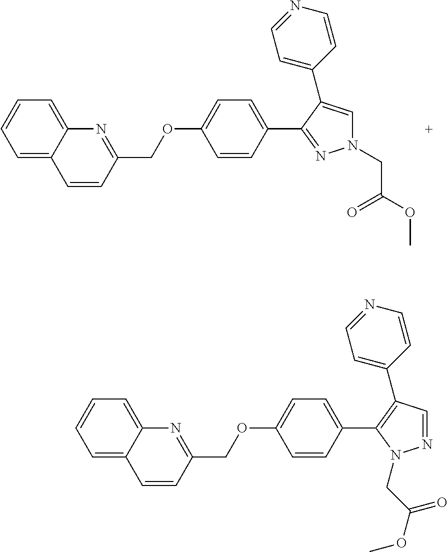

To a stirred solution of 2-[4-(4-pyridin-4-yl-1H-pyrazol-3-ye-phenoxymethyl]-quinoline (0.300 g, 0.793 mmol) in DMF (6 mL) were added ethyl bromoacetate (0.09 mL, 0.951 mmol) and cesium carbonate (0.775 g, 2.378 mmol). The mixture was stirred at room temperature for 3 h. Then it was quenched with water and extracted with EtOAc. The organic solvents were separated, dried over sodium sulphate and evaporated to dryness in vacuo. The crude residue was purified by column chromatography (silicagel; EtOAc/methanol 100/0 to 95/5). The desired fractions were collected and evaporated in vacuo to yield a mixture of the two regioisomers I-3 and I-3′, 60% pure that was used for the next reaction without further purification (0.280 g, 47%).

### Intermediate 4

### 2-{4-Pyridin-4-yl-3-[4-(quinolin-2-ylmethoxy)-phenyl]-pyrazol-1-yl}-ethanol (I-4)

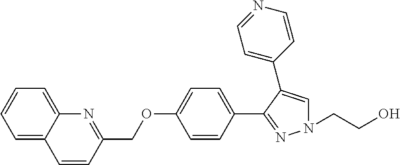

To a stirred solution of the mixture of intermediates I-3 and I-3′ (0.280 g, 0.379 mmol) in a mixture of DCM (4 mL) and methanol (1 mL) was added sodium borohydride (0.072 g, 1.896 mmol). The reaction mixture was stirred at room temperature for 2 h.

The mixture was then quenched with water, extracted with more DCM, the organic solvent was dried over sodium sulphate and evaporated to dryness. The crude residue was purified by column chromatography (silicagel; EtOAc/methanol 100/0 to 90/10). The desired fractions were collected and evaporated in vacuo to yield I-4 (0.080 g, 50%). ¹H NMR (400 MHz, DMSO-d₆) δ ppm 3.81 (q, J=5.3 Hz, 2 H), 4.19 (t, J=5.5 Hz, 2 H), 4.98 (t, J=5.3 Hz, 1H), 5.39 (s, 2 H), 7.08-7.13 (m, 2 H), 7.19-7.24 (m, 2 H), 7.30-7.38 (m, 2 H), 7.59-7.66 (m, 1H), 7.71 (d, J=8.6 Hz, 1H), 7.79 (ddd, J=8.4, 6.9, 1.5 Hz, 1H), 8.01 (d, J=7.9 Hz, 1H), 8.03 (d, J=7.9 Hz, 1H), 8.16 (s, 1H), 8.41-8.46 (m, 3 H). C₂₆H₂₂N₄O₂ LCMS: Rt 3.8, m/z 423 [M+H]⁺.

The corresponding regioisomer I-4′ 2-{4-Pyridin-4-yl-5-[4-(quinolin-2-ylmethoxy)-phenyl]-pyrazol-1-yl}-ethanol was also isolated from the chromatographic separation (0.030 g, 18.7%). C₂₆H₂₂N₄O₂.

### Intermediate 5

### Methanesulfonic acid 2-{4-Pyridin-4-yl-3-[4-(quinolin-2-ylmethoxy)-phenyl]-pyrazol-1-yl}-ethyl ester (I-5)

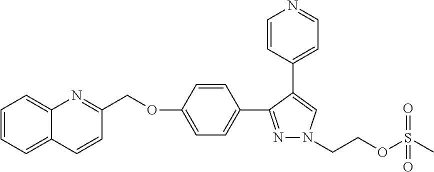

To a solution of I-4 (5 mg, 11.8 μmol) in dichloromethane (1 mL) was added pyridine (11 μL, 136.7 μmol) and this solution was stirred at 0° C. Methane sulfonyl chloride (7 μL, 90.4 μmol) was then added and the stirring was continued for 4 hours at 0° C. after which the solvent was evaporated by flushing with nitrogen. The crude mixture was redissolved in methanol (0.5 mL), diluted with water (4.5 mL) and passed through a C₁₈ SepPak® cartridge (Waters) that was preconditioned with methanol (3 mL) and milliQ® water (Millipore) (6 mL). The cartridge was then rinsed three times with an additional volume of water (2 mL) to remove unreacted methane sulfonyl chloride. The product was eluted from the cartridge using acetonitrile (3 mL) and the solvents were evaporated under reduced pressure. Prior to the evaporation of solvents, HPLC analysis was performed to examine the conversion of precursor I-4 to its O-mesyl derivative. This HPLC analysis was done on an analytical XTerra™ RP C₁₈ column (Waters), which was eluted with gradient mixtures of water and acetonitrile (0 min: 95:5 v/v, 25 min: 10:90 v/v, 30 min: 10:90 v/v, linear gradient) at a flow rate of 1 mL/min. The analysis showed that the conversion rate was between 85 and 98% (n=16). Residual water was removed by azeotropic distillation with acetonitrile and the mixture was dried in the vacuum oven. On the day of radiolabeling experiments, which was usually the next morning, this reaction product was dissolved in anhydrous DMF (1.5 mL) and used (0.3 mL) for the direct nucleophilic radiofluorination reaction.

### B. Preparation of the Final Compounds

### Example B1

### 2-{4-[1-(3-Fluoropropyl)-4-pyridin-4-yl-1H-1-pyrazol-3-yl]-phenoxymethyl}-quinoline (B-1)

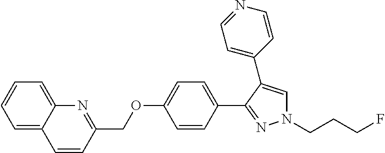

A mixture of 2-[4-(4-pyridin-4-yl-1H-pyrazol-3-yl)-phenoxymethyl]-quinoline (0.500 g, 1.321 mmol), 3-fluoropropan-1-ol (0.155 g, 1.982 mmol), diisopropyl azodicarboxylate (0.393 mL, 1.982 mmol) and triphenylphosphine (0.520 g, 1.982 mmol) in THF (3 mL) was heated in a microwave oven at 120° C. for 20 min. After this time the solvent was evaporated to dryness in vacuo and the crude residue was purified by column chromatography (silicagel; first with heptane/THF 80/20 to 50/50 and then with DCM/EtOAc 100/0 to 0/100). The desired fractions were collected and evaporated in vacuo to yield the desired compound B-1 (0.190 g, 32.8%) as a white solid, m.p. 101.9° C.

¹H NMR (400 MHz, CDCl₃) δ ppm 2.25-2.43 (m, 2 H), 4.33 (t, J=6.7 Hz, 2 H), 4.50 (dt, J=47.2, 5.5 Hz, 2 H), 5.41 (s, 2 H), 6.95-7.07 (m, 2 H), 7.13-7.21 (m, 2 H), 7.35-7.43 (m, 2 H), 7.52-7.60 (m, 1H), 7.63 (s, 1H), 7.69 (d, J=8.3 Hz, 1H), 7.74 (ddd, J=8.5, 7.0, 1.4 Hz, 1H), 7.84 (d, J=8.1 Hz, 1H), 8.08 (d, J=8.6 Hz, 1H), 8.20 (d, J=8.6 Hz, 1H), 8.48 (br. d, J=6.0 Hz, 2 H). C₂₇H₂₃FN₄O. LCMS: Rt 4.4, m/z 439 [M+H]⁺.

The corresponding regioisomer B-1′ 2-{4-[2-(3-fluoropropyl)-4-pyridin-4-yl-2H-pyrazol-3-yl]-phenoxymethyl}-quinoline was also isolated from the chromatographic separation (0.100 g, 17.3%) as a white powder. C₂₂H₂₃FN₄O.

Radiosynthesis: Production of [¹⁸F]fluoride and [¹⁸F]B-1

[¹⁸F]fluoride ([¹⁸F]F⁻) was produced by an [¹⁸O(p,n)¹⁸F] reaction by irradiation of 2 mL of 97% enriched [¹⁸O]H₂O (Rotem HYOX18, Rotem Industries, Beer Sheva, Israel) in a niobium target using 18 MeV protons. After irradiation, the resultant [¹⁸F]F⁻ was separated from [¹⁸O]H₂O using a SepPak™ Light Accell plus QMA anion exchange cartridge (Waters), which was preconditioned by successive treatments with 0.5 M K₂CO₃ solution (10 mL) and water (2×10 mL). The [¹⁸F]F⁻ was then eluted from the cartridge into a conical reaction vial (1 mL) using a solution containing 2.47 mg of K₂CO₃ and 27.9 mg of Kryptofix® 222 in 0.75 mL of H₂O/CH₃CN (5:95 v/v) and the solvents were evaporated at 80° C. by applying microwave power of 35 W for 360 seconds. After evaporation of the solvent, [¹⁸F]F⁻ was further dried by azeotropic distillation using acetonitrile (1 mL) that was added in two portions. The conditions used for this drying process were power 35 W, temperature 70° C., time 180 seconds (1st drying step) and 360 seconds (2nd drying step). The radiolabeling precursor I-2; ˜0.6 mg in 0.3 mL DMF) was then added to this dried [¹⁸F]F⁻/K₂CO₃/Kryptofix® 222 complex and the nucleophilic substitution reaction was carried out by using microwave assisted heating at 120° C. for 60 seconds (35 W power). After the reaction, the crude mixture was diluted with 0.6 mL of water and injected on to the HPLC system consisting of an XBridge™ column (C₁₈, 5 μm; 4.6 mm×150 mm; Waters) that was eluted with a mixture of 0.05 M sodium acetate buffer (pH 5.5) and EtOH (60:40 v/v) at a flow rate of 1.0 mL/min. UV detection of the HPLC eluate was performed at 254 nm. The radiolabeled product [¹⁸F]B-1 was collected at 22 min. The collected peak corresponding to the [¹⁸F]B-1 was then diluted with normal saline (Mini Plasco®, Braun, Melsungen, Germany) to reduce the ethanol concentration to <5% and sterile filtered through a 0.22 μm membrane filter (Millex®-GV, Millipore, Ireland). The purity of this HPLC purified radiotracer was analyzed using an analytical HPLC system consisting of an XBridg™ column (C₁₈, 3.5 μm; 3 mm 100 mm; Waters) eluted with a mixture of 0.05 M sodium acetate buffer pH 5.5 and acetonitrile (60:40 v/v) at a flow rate of 0.8 mL/min (Rt=6.1 min). The mean decay-corrected radiochemical yield was between 15 and 27% (relative to starting [¹⁸F]F⁻, n=3). The radiochemical purity as examined using the above analytical HPLC system was >99%. The average specific radioactivity of the tracer was found to be 322 GBq/μmol (n=3) at the end of synthesis.

### Example B2

### 2-{4-[1-(2-Fluoroethyl)-4-pyridin-4-yl-1H-1-pyrazol-3-yl]-phenoxymethyl}-quinoline (B-2)

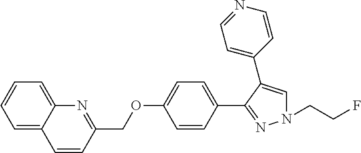

A mixture of 2-[4-(4-pyridin-4-yl-1H-pyrazol-3-yl)-phenoxymethyl]-quinoline (0.120 g, 0.317 mmol), 1-bromo-2-fluoroethane (0.047 mL, 0.634 mmol) and cesium carbonate (0.310 g, 0.951 mmol) in DMF (3 mL) was heated in a microwave oven at 150° C. for 10 min. After this time the solvent was evaporated to dryness in vacuo and the crude residue was purified by column chromatography (silicagel; first with heptane/THF 80/20 to 50/50 and then with DCM/EtOAc 100/0 to 0/100). The desired fractions were collected and evaporated in vacuo to yield the desired compound B2 (0.060 g, 44.6%) as a white solid, m.p. 110.8° C. ¹H NMR (500 MHz, CDCl₃) δ ppm 4.45 (dt, J=26.9, 4.6 Hz, 2 H), 4.82 (ddd, J=47.0, 4.6, 4.5 Hz, 2 H), 5.40 (s, 2 H), 7.02 (d, J=8.7 Hz, 2 H), 7.15-7.20 (m, 2 H), 7.40 (d, J=8.7 Hz, 2 H), 7.51-7.57 (m, 1H), 7.60 (d, J=9.0 Hz, 1H), 7.68 (s, 1H), 7.70-7.76 (m, 1H), 7.82 (d, J=7.8 Hz, 1H), 8.08 (d, J=8.4 Hz, 1H), 8.18 (d, J=8.7 Hz, 1H), 8.44-8.51 (m, 2 H). C₂₆H₂₁FN₄O. LCMS: Rt 4.2, m/z 425 [M+H]⁺.

The corresponding regioisomer B2′ 2-{4-[2-(2-fluoroethyl)-4-pyridin-4-yl-2H-pyrazol-3-yl]phenoxymethyl}-quinoline was also isolated from the chromatographic separation (0.020 g, 14.9%) as a white powder. C₂₆H₂₁FN₄O.

Radiosynthesis: Production of [¹⁸F]fluoride and [¹⁸F]B-2 [¹⁸F]fluoride ([¹⁸F]F⁻) was produced by an [¹⁸O(p,n)¹⁸F] reaction by irradiation of 2 mL of 97% enriched [¹⁸O]H₂O (Rotem HYOX18, Rotem Industries, Beer Sheva, Israel) in a niobium target using 18 MeV protons. After irradiation, the resultant [¹⁸F]F⁻ was separated from [¹⁸O]H₂O using a SepPak® Light Accell plus QMA anion exchange cartridge (Waters), which was preconditioned by successive treatments with 0.5 M K₂CO₃ solution (10 mL) and water (2×10 mL). The [¹⁸F]F⁻ was then eluted from the cartridge into a conical reaction vial (1 mL) using a solution containing 2.47 mg of K₂CO₃ and 27.9 mg of Kryptofix® 222 in 0.75 mL of H₂O/CH₃CN (5:95 v/v) and the solvents were evaporated at 110° C. by applying conventional heating for 2 min. After evaporation of the solvent, [¹⁸F]F⁻ was further dried by azeotropic distillation using acetonitrile (1 mL) at a temperature of 110° C. for 6 min. The radiolabeling precursor I-5, ˜0.6 mg in 0.3 mL DMF) was then added to this dried [¹⁸F]F⁻/K₂CO₃/Kryptofix® 222 complex and the nucleophillic substitution reaction was carried out by conventional heating at 90° C. for 10 min. After the reaction, the crude mixture was diluted with 1.4 mL of water and injected on to the HPLC system consisting of an XBridge™ column (C18, 5 μm; 4.6 mm×150 mm; Waters) that was eluted with a mixture of 0.05 M sodium acetate buffer (pH 5.5) and EtOH (65:35 v/v) at a flow rate of 1.2 mL/min. UV detection of the HPLC eluate was performed at 254 nm. The radiolabeled product [¹⁸F]B-2 was collected at 43 min. The collected peak corresponding to the [¹⁸F]B-2 was then diluted with normal saline (Mini Plasco®, Braun, Melsungen, Germany) to reduce the ethanol concentration to <5% and sterile filtered through a 0.22 μm membrane filter (Millex®-GV, Millipore, Ireland). The purity of this HPLC purified radiotracer was analyzed using an analytical HPLC system consisting of an XBridge™ column (C18, 3.5 μm; 3 mm×100 mm; Waters) eluted with a mixture of 0.05 M sodium acetate buffer pH 5.5 and acetonitrile (60:40 v/v) at a flow rate of 0.8 mL/min (Rt=3.9 min) The mean decay-corrected radiochemical yield was 17±5% (relative to starting [¹⁸F]F⁻, n=16). The radiochemical purity as examined using the above analytical HPLC system was >99%. The average specific radioactivity of the tracer was found to be 315 GBq/μmol (n=16) at the end of synthesis.

### C. Analytical Part

### Melting Points:

Values are peak values, and are obtained with experimental uncertainties that are commonly associated with this analytical method.

For a number of compounds, melting points were determined in open capillary tubes on a Mettler FP62 apparatus. Melting points were measured with a temperature gradient of 10° C./minute. Maximum temperature was 300° C. The melting point was read from a digital display.

### LCMS-Methods:

For LCMS-characterization of the compounds of the present invention, the following methods were used.

### General Procedure A

The HPLC measurement was performed using a HP 1100 from Agilent Technologies comprising a pump (quaternary or binary) with degasser, an autosampler, a column oven, a diode-array detector (DAD) and a column as specified in the respective methods below. Flow from the column was split to a MS spectrometer. The MS detector was configured either with an electrospray ionization source or an ESCI dual ionization source (electrospray combined with atmospheric pressure chemical ionization). Nitrogen was used as the nebulizer gas. The source temperature was maintained at 140° C. Data acquisition was performed with MassLynx-Openlynx software.

### General Procedure B

The HPLC measurement was performed using a HP 1100 from Agilent Technologies comprising a binary pump with degasser, an autosampler, a column oven, a diode-array detector (DAD) and a column as specified in the respective methods below. Flow from the column was split to a MS spectrometer. The MS detector was configured with an ESCI dual ionization source (electrospray combined with atmospheric pressure chemical ionization). Nitrogen was used as the nebulizer gas. The source temperature was maintained at 100° C. Data acquisition was performed with Chemsation-Agilent Data Browser software.

### Method 1, for Compounds I-1, B-2

In addition to the general procedure: Reversed phase HPLC was carried out on an XDB-C18 cartridge (1.8 μm, 2.1×30 mm) from Agilent, at 60° C. with a flow rate of 1 ml/min, at 60° C. The gradient conditions used are: 90% A (0.5 g/l ammonium acetate solution), 5% B (acetonitrile), 5% C (methanol) to 50% B and 50% C in 6.5 minutes, to 100% B at 7 minutes and equilibrated to initial conditions at 7.5 minutes until 9.0 minutes. Injection volume 2 High-resolution mass spectra (Time of Flight, TOF) were acquired only in positive ionization mode by scanning from 100 to 750 in 0.5 seconds using a dwell time of 0.1 seconds. The capillary needle voltage was 2.5 kV and the cone voltage was 20 V. Leucine-Enkephaline was the standard substance used for the lock mass calibration.

### Method 2, for Compound I-2

In addition to the general procedure: Reversed phase HPLC was carried out on an XDB-C18 cartridge (1.8 μm, 2.1×30 mm) from Agilent, with a flow rate of 0.8 ml/min, at 60° C. The gradient conditions used are: 95% A (0.5 g/l ammonium acetate solution+5% acetonitrile), 5% B (mixture of acetonitrile/methanol, 1/1), kept 0.2 minutes, to 100% B in 3.0 minutes, kept for 3.15 minutes and equilibrated to initial conditions at 3.3 minutes until 5.0 minutes. Injection volume 2 μl. Low-resolution mass spectra (Quadrupole, MSD) were acquired in electrospray mode by scanning from 100 to 1000 in 0.99 seconds, step size of 0.30 and peak width of 0.10 minutes. The capillary needle voltage was 1.0 kV and the fragmentor voltage was 70V for both positive and negative ionization modes.

### Method 3, for Compound I-4

In addition to the general procedure: Reversed phase HPLC was carried out on a Sunfire-C18 column (2.5 μm, 2.1×30 mm) from Waters, with a flow rate of 1.0 ml/min, at 60° C. The gradient conditions used are: 95% A (0.5 g/l ammonium acetate solution+5% of acetonitrile), 2.5% B (acetonitrile), 2.5% C (methanol) to 50% B, 50% C in 6.5 minutes, kept for 7.0 minutes and equilibrated to initial conditions at 7.3 minutes until 9.0 minutes. Injection volume 2 μl. High-resolution mass spectra (Time of Flight, TOF) were acquired by scanning from 100 to 750 in 0.5 seconds using a dwell time of 0.3 seconds. The capillary needle voltage was 2.5 kV for positive ionization mode and 2.9 kV for negative ionization mode. The cone voltage was 20 V for both positive and negative ionization modes. Leucine-Enkephaline was the standard substance used for the lock mass calibration.

### Method 4, for Compound B-1

In addition to the general procedure: Reversed phase HPLC was carried out on a XDB-C18 cartridge (1.8 μm, 2.1×30 mm) from Agilent, with a flow rate of 0.8 ml/min, at 60° C. The gradient conditions used are: 90% A (0.5 g/l ammonium acetate solution), 10% B (mixture of Acetonitrile/Methanol, 1/1), to 100% B in 6.0 minutes, kept for 6.5 minutes and equilibrated to initial conditions at 7.0 minutes until 9.0 minutes. Injection volume 2 μl. Low-resolution mass spectra (SQD detector; quadrupole) were acquired in positive ionization mode by scanning from 100 to 1000 in 0.1 seconds using an inter-channel delay of 0.08 second. The capillary needle voltage was 3 kV. The cone voltage was 20 V and 50V for positive ionization mode and 30V for negative ionization mode.

High-performance liquid chromatography (HPLC) analysis was performed on a LaChrom Elite HPLC system (Hitachi, Darmstadt, Germany) connected to a UV spectrometer set at 254 nm. For the analysis of radiolabeled compounds, the HPLC eluate after passage through the UV detector was led over a 3 in. NaI(Tl) scintillation detector connected to a single channel analyzer (Gabi box, Raytest, Straubenhardt Germany). The radioactivity measurements during biodistribution studies and in vivo stability analyses were done using an automatic γ-counter (with a 3 in. NaI(Tl) well crystal) coupled to a multichannel analyzer (Wallac 1480 Wizard 3″, Wallac, Turku, Finland). All reagents and solvents were obtained commercially from Acros Organics (Geel, Belgium), Aldrich, Fluka, Sigma (Sigma-Aldrich, Bornem, Belgium), or Fischer Bioblock Scientific (Tournai, Belgium) and used as supplied. All animal experiments were conducted with the approval of the institutional ethical committee for conduct of experiments on animals.

### Nuclear Magnetic Resonance (NMR)

¹H NMR spectra were recorded either on a Bruker DPX-400 or on a Bruker AV-500 spectrometer with standard pulse sequences, operating at 400 MHz and 500 MHz respectively, using CDCl₃ and DMSO-d₆ as solvents. Chemical shifts (6) are reported in parts per million (ppm) downfield from tetramethylsilane (TMS), which was used as internal standard.

### D. Studies with [¹⁸F]B-1

### D.1. Biodistribution Studies

The biodistribution study of [¹⁸F]B-1 was carried out in male Wistar rats (body weight 250-350 g) at 2 min, 30 min and 60 min post injection (p.i.) (n=3/time point). Rats were injected with about 1.1 MBq of the tracer via tail vein under anesthesia (2.5% Isoflurane in O₂ at 1 L/min flow rate) and sacrificed by decapitation at above specified time points. Blood and major organs were collected in tared tubes and weighed. The radioactivity in blood, organs and other body parts was measured using an automated gamma counter The distribution of radioactivity in different parts of the body at different time points p.i. of the tracer was calculated and expressed as percentage of injected dose (% ID), and as percentage of injected dose per gram tissue (% ID/g) for the selected organs. % ID is calculated as cpm in organ/total cpm recovered. For calculation of total radioactivity in blood, blood mass was assumed to be 7% of the body mass.

The results of the in vivo distribution study of [¹⁸F]B-1 in male Wistar rats is presented in Tables 1 and 2. Table 1 shows the % injected dose (% ID) values at 2 min, 30 min and 60 min postinjection (p.i.) of the radiotracer. At 2 min p.i. about 3.4% of the injected dose was present in the blood, and this cleared to 1.9% by 60 min after injection of the tracer. The total brain uptake of the tracer at 2 min p.i. was ˜0.5%, with 0.4% of the ID in the cerebrum and ˜0.1% in the cerebellum, suggesting a rather lower (initial) uptake of [¹⁸F]B-1 in the brain. 0.015% ID was present in the striatum, which has the highest expression of PDE10A enzyme, and where the radiotracer is expected to show binding. At 60 min p.i. 1.9% of ID was still present in the blood indicating rather poor clearance of this tracer from the blood circulation. The compound was cleared mainly by hepatobiliary system as there was in total 56% of ID present in the liver and intestines at 60 min after injection of the radiotracer.

TABLE 1
Biodistribution of [¹⁸F]B-1 in normal rats at 2, 30 and 60 min p.i.

| | % ID ^{a} | | |
|---|---|---|---|
| Organ | 2 min | 30 min | 60 min |
| Urine | 0.09 ± 0.0 | 0.61 ± 0.3 | 1.87 ± 0.2 |
| Kidneys | 5.31 ± 0.7 | 1.55 ± 0.1 | 1.45 ± 0.1 |
| Liver | 48.20 ± 1.8 | 49.64 ± 5.3 | 41.44 ± 1.5 |
| Spleen + Pancreas | 1.63 ± 0.1 | 0.42 ± 0.1 | 0.31 ± 0.0 |
| Lungs | 2.79 ± 0.6 | 1.81 ± 0.0 | 0.32 ± 0.0 |
| Heart | 0.87 ± 0.1 | 0.22 ± 0.0 | 0.17 ± 0.0 |
| Stomach | 1.74 ± 0.2 | 4.60 ± 0.6 | 10.00 ± 2.5 |
| Intestines | 8.89 ± 0.5 | 14.44 ± 3.0 | 14.72 ± 4.1 |
| Striatum | 0.015 ± 0.00 | 0.014 ± 0.00 | 0.015 ± 0.00 |
| Hippocampus | 0.013 ± 0.00 | 0.004 ± 0.00 | 0.003 ± 0.00 |
| Cortex | 0.072 ± 0.01 | 0.018 ± 0.00 | 0.012 ± 0.00 |
| Rest of cerebrum | 0.307 ± 0.05 | 0.122 ± 0.00 | 0.071 ± 0.01 |
| Cerebrum total | 0.407 ± 0.1 | 0.158 ± 0.01 | 0.101 ± 0.01 |
| Cerebellum | 0.096 ± 0.0 | 0.021 ± 0.0 | 0.012 ± 0.00 |
| Blood | 3.37 ± 0.3 | 2.12 ± 0.1 | 1.94 ± 0.1 |
| Carcass | 27.70 ± 0.7 | 25.24 ± 1.7 | 27.96 ± 0.7 |

Data are expressed as mean ± SD; n = 3 per time point;
^{a} Percentage of injected dose calculated as cpm in organ/total cpm recovered

Because of its lipophilic character, the urinary excretion of the tracer was minimal with only ˜3.3% ID present in the urinary system at 60 min p.i. In view of the large mass of the carcass, significant amount of the injected dose (˜26% ID) was present in the carcass at all time points examined. Typically, carcass constitutes to >90% of the total body mass of the animal. Table 2 shows the % ID/g values for different organs at 2 min, 30 min and 60 min p.i.

TABLE 2
[¹⁸F]B-1 concentration in different organs at 2, 30 and 60 min p.i.

| | % ID/g ^{a} | | |
|---|---|---|---|
| Organ | 2 min | 30 min | 60 min |
| Kidneys | 2.22 ± 0.29 | 0.64 ± 0.04 | 0.46 ± 0.02 |
| Liver | 3.79 ± 0.19 | 4.00 ± 0.37 | 3.11 ± 0.06 |
| Spleen + Pancreas | 1.18 ± 0.17 | 0.27 ± 0.01 | 0.19 ± 0.01 |
| Lungs | 2.36 ± 0.75 | 1.38 ± 0.11 | 0.20 ± 0.02 |
| Heart | 1.06 ± 0.20 | 0.26 ± 0.02 | 0.16 ± 0.00 |
| Striatum | 0.40 ± 0.05 | 0.41 ± 0.00 | 0.25 ± 0.01 |
| Hippocampus | 0.20 ± 0.02 | 0.08 ± 0.01 | 0.05 ± 0.01 |
| Cortex | 0.40 ± 0.11 | 0.11 ± 0.02 | 0.06 ± 0.00 |
| Rest of cerebrum | 0.30 ± 0.04 | 0.12 ± 0.01 | 0.07 ± 0.00 |
| Cerebrum total | 0.31 ± 0.05 | 0.12 ± 0.01 | 0.07 ± 0.00 |
| Cerebellum | 0.39 ± 0.06 | 0.09 ± 0.01 | 0.04 ± 0.00 |
| Blood | 0.19 ± 0.02 | 0.12 ± 0.01 | 0.08 ± 0.00 |
| Cerebrum + | 0.33 ± 0.05 | 0.12 ± 0.01 | 0.07 ± 0.00 |
| Cerebellum | | | |

Data are expressed as mean ± SD; n = 3 per time point;
^{a} % ID/g values are calculated as % ID/weight of the organ in g.

As kidneys and liver are the excretory organs, they have the highest % ID/g values with about 2.2% ID/g for kidneys and 3.8% ID/g for liver at 2 min p.i. The % ID/g values for different regions of brain, namely striatum, hippocampus, cortex and cerebellum are presented in Table 2. At 2 min p.i. the radioactivity concentration in the striatum was about 0.4% ID/g, and similar concentration was present in the cortex as well as in the in the cerebellum where the expression of PDE10A receptors is minimal. In hippocampus there was about 0.2% ID/g. At 60 min p.i., the radioactivity concentration in the hypocampus, cortical regions and the cerebellum was <0.07% ID/g, while the intensity of the radiotracer in the striatum at 30 min p.i. was essentially the same as that of 2 min p.i., and was about 0.25% ID/g tissue at 60 min p.i. This indicates the slower washout of [¹⁸F]B-1 from the striatum, and is consistent with the higher expression of the PDE10A in this region.

The % ID/g tissue values were normalized for their body weight in order to correct for differences in body weight between different animals. The normalized values are presented in Table 3.

TABLE 3
[¹⁸F]B-1 concentration in different organs at 2, 30
and 60 min p.i. normalized for the body weight of the animal

| | % ID/g × kg ^{a} | | |
|---|---|---|---|
| Organ | 2 min | 30 min | 60 min |
| Kidneys | 0.57 ± 0.07 | 0.16 ± 0.01 | 0.16 ± 0.01 |
| Liver | 0.97 ± 0.05 | 1.03 ± 0.08 | 1.04 ± 0.02 |
| Spleen + Pancreas | 0.30 ± 0.04 | 0.07 ± 0.00 | 0.06 ± 0.01 |
| Lungs | 0.60 ± 0.19 | 0.35 ± 0.03 | 0.07 ± 0.01 |
| Heart | 0.27 ± 0.05 | 0.07 ± 0.00 | 0.05 ± 0.00 |
| Striatum | 0.102 ± 0.01 | 0.106 ± 0.00 | 0.083 ± 0.01 |
| Hippocampus | 0.052 ± 0.01 | 0.021 ± 0.00 | 0.016 ± 0.00 |
| Cortex | 0.103 ± 0.03 | 0.027 ± 0.01 | 0.019 ± 0.00 |
| Rest of cerebrum | 0.077 ± 0.01 | 0.030 ± 0.00 | 0.022 ± 0.00 |
| Cerebrum total | 0.080 ± 0.01 | 0.032 ± 0.00 | 0.024 ± 0.00 |
| Cerebellum | 0.100 ± 0.02 | 0.023 ± 0.00 | 0.015 ± 0.00 |
| Blood | 0.05 ± 0.00 | 0.03 ± 0.00 | 0.03 ± 0.00 |
| Cerebrum + | 0.08 ± 0.01 | 0.03 ± 0.00 | 0.02 ± 0.00 |
| Cerebellum | | | |

Data are expressed as mean ± SD; n = 3 per time point;
^{a} % ID/g × kg values are calculated as % ID/weight of the organ in g multiplied by the body weight of the animal in kg

TABLE 4
Clearance of [¹⁸F]B-1 from different regions of the brain calculated
as 2 min to 30 min or 2 min to 60 min ratio for % ID/g values

| Brain region | 2 min/30 min | 2 min/60 min |
|---|---|---|
| Striatum | 0.97 | 1.62 |
| Hippocampus | 2.54 | 4.33 |
| Cortex | 3.81 | 7.19 |
| Rest of cerebrum | 2.54 | 4.53 |
| Cerebrum total | 2.54 | 4.34 |
| Cerebellum | 4.37 | 8.81 |
| Blood | 1.60 | 2.28 |

Table 4 shows 2 min/30 min and 2 min/60 min ratios for % ID/g values for different regions of the brain. For the 2 min/60 min ratios, the cerebellum has the highest ratio of 8.8, indicating that the clearance of the (initial) radioactivity is the fastest from this region, followed by other regions of the brain such as cortical areas and the hippocampus, which do not express PDE10A. On the other hand, striatum has a ratio of 0.97 at 30 min, suggesting that the activity in this organ remained at same level as that of 2 min p.i. However, by 60 min p.i. this ratio increased to 1.6 due to the clearance of the tracer. The results from these biodistribution studies indicate that although the initial uptake was rather low, the washout from the striatum is much slower when compared to the reference region cerebellum. This suggests the specific retention of [¹⁸F]B-1 in the PDE10A-rich region striatum.

TABLE 5
Clearance of [¹⁸F]B-1 from different regions of the brain
calculated as 2 min to 30 min or 2 min to 60 min ratio for
% ID/g values normalized for the body weight of the animal.

| Brain region | 2 min | 30 min | 60 min |
|---|---|---|---|
| striatum/hippocampus | 2.0 | 5.1 | 5.2 |
| striatum/cortex | 1.0 | 3.9 | 4.4 |
| striatum/cerebellum | 1.0 | 4.6 | 5.6 |
| striatum/blood | 2.1 | 3.5 | 3.0 |

Table 5 presents the ratios between striatum and other regions of the brain as well as blood at different time points post injection of [¹⁸F]B-1. Striatum was considered as a PDE10-rich region and cerebellum as a reference region. Therefore, high striatum to cerebellum ratio is desired in order to have good quality images in vivo. At 2 min p.i., the striatum to cerebellum ratio was about 1.0 and this ratio increased to 5.6 by 60 min after injection of the tracer. Striatum to cortex and striatum to hippocampus ratios were also >4.4 confirming the specific retention of [¹⁸F]B-1 in the striatum.

### D.2. Plasma Metabolite Analysis

The metabolic stability of [¹⁸F]B-1 was studied in normal rats by determination of the relative amounts of parent tracer and metabolites in the blood at 1 hour p.i. of the tracer. After intravenous (i.v.) administration of about 19 MBq [¹⁸F]B-1 via tail vein under anesthesia (2.5% Isoflurane in O₂ at 1 L/min flow rate), rats were sacrificed by decapitation at 60 min p.i. (n=2). Blood was collected in heparin containing tubes (4.5 mL LH PST tubes; BD vacutainer, BD, Franklin Lakes, N.J., USA) and immediately centrifuged for 10 min at 3000 rpm to separate the plasma. About 0.4 mL of this supernatant plasma was spiked with about 5 μg of non-radioactive B-1 (1 mg/mL solution) and injected on to HPLC, which was connected with a Chromolith performance column (C18, 3 mm×100 mm, Merck KGaA, Darmstadt, Germany). The mobile phase consisted of 0.05 M NaOAc pH 5.5 (solution A) and acetonitrile (solvent B). The following method was used for the analysis: isocratic elution with 100% A for 4 min at a flow rate of 0.5 mL/min, then linear gradient to 90% B by 14 min at a flow rate of 1 mL/min, and isocratic elution with mixture of 10% A and 90% B until 17 min. After passing through UV detector (254 nm), the HPLC eluate was collected as 1-mL fractions using an automatic fraction collector and the radioactivity of these fractions was measured in the γ-counter.

The metabolic stability was assessed in plasma collected from normal rats (n=2) 60 min post injection of [¹⁸F]B-1 using RP-HPLC. The cold intact compound B-1 was co-injected onto HPLC to identify the intact parent tracer. An overview of the results from this plasma analysis is presented in Table 6.

TABLE 6
Relative percentages of intact tracer and metabolites
in rat plasma at 60 min. p.i. of [¹⁸F]B-1.

| (%) | mean ± SD (n = 2) |
|---|---|
| Polar metabolite(s) | 46.9 ± 5.7 |
| Intact tracer | 51.5 ± 4.1 |
| Apolar metabolite(s) | 1.6 ± 1.5 |

The analysis showed that at 60 min post injection of the radiotracer, about 51% of the recovered radioactivity was in the form of intact tracer and about 47% of the activity was in the form of polar metabolites. The radioactivity corresponding to the (lipophilic) fractions eluting after the intact tracer was negligible (˜2%).

### D.3. Brain Metabolite Analysis

From the same animals that were used for plasma analysis, brain was also dissected and rinsed with saline (Mini Plasco®, Braun, Melsungen, Germany) to rinse off the blood. Cerebrum and cerebellum were collected separately and homogenized in 3 mL and 2 mL of acetonitrile, respectively, for about 3 min. A volume of 1 mL of this homogenate was diluted with an equal volume of water and a part of this homogenate was filtered through 0.22 μm filter (Millex®-GV, Millipore, Ireland). About 0.5 mL of the filtrate was spiked with 5 μL of non-radioactive B-1 solution (1 mg/mL) and then injected on to HPLC, which was connected with an XBridge™ column (C18, 5 μM, 3 mm×100 mm, Waters) eluted with mixtures of 0.05 M sodium acetate buffer pH 5.5 and acetonitrile (60:40 v/v) at a flow rate of 0.8 mL/min. Similar to the plasma analysis, the HPLC eluate was collected as 1-mL fractions after passing through UV detector, and the radioactivity in the fractions was measured using the automated gamma counter.

The above plasma analysis indicates that [¹⁸F]B-1 is metabolized to more polar radiometabolites. Based on their higher hydrophilicity we can expect that these radiometabolites may not cross the blood brain barrier (BBB). However, in order to rule out the presence or entry of these metabolites to the brain, we have also analyzed cerebrum and cerebellum samples of rats after isolation and subsequent homogenization at 60 min after injection of [¹⁸F]B-1 (n=2).

TABLE 7
Relative percentages of intact tracer and metabolites in
rat cerebrum and cerebellum at 60 min p.i. of [¹⁸F]B-1.

| (%) | Cerebrum | Cerebellum |
|---|---|---|
| Polar metabolite(s) | 6.6 ± 2.9 | 15.3 ± 1.8 |
| Intact tracer | 93.2 ± 2.8 | 83.3 ± 1.0 |
| Apolar metabolites | 0.3 ± 0.0 | 1.4 ± 0.9 |

Results are presented as mean ± SD (n = 2)

Table 7 gives the overview of the results of the radiometabolite assay in the cerebrum as well as cerebellum at 60 min after injection of [¹⁸F]B-1. About 93% of the recovered radioactivity from the cerebrum homogenate analysis was in the form of the parent tracer [¹⁸F]B-1. In the cerebellum tissue about 83% of the recovered radioactivity was present as the intact tracer. Since the rat was not perfused, the presence of polar metabolites in cerebrum (˜7%) and in cerebellum (˜15%) could be (partly) due to the blood that was still present in brain. The fraction of apolar metabolites in the brain was negligible.

D.4. microPET Imaging Study

Imaging experiments were performed on a Focus™ 220 microPET scanner (Concorde Microsystems, Knoxville, Tenn., USA) using male Wistar rats with body weight between 200 and 300 g. Animals were injected with about 74 MBq of high specific activity formulation of [¹⁸F]B-1 via tail vein under anesthesia (2.5% Isoflurane in O₂ at 1 L/min flow rate). Dynamic scans of 90 min were acquired in List mode. Acquisition data were Fourier rebinned in different time frames (4×15 s, 4×1 min, 5×3 min, 14×5 min) and reconstructed with Filtered Back Projection (FBP). An average image (frame 1 to 27) of the reconstructed data was spatially normalized to an in-house created [¹¹C]raclopride template of the rat brain in Paxinos coordinates. The affine transformation was then used to normalize all time frames of the dynamic dataset to allow automated and symmetric volumes of interest (VOIs) analyses. Time-activity curves (TAC) were generated for striatum, cortex and cerebellum for each individual scan, using PMOD software (v 3.0, PMOD Technologies Ltd., Zurich, Switzerland). The radioactivity concentration in the different brain regions was expressed as standardized uptake value (SUV) as a function of time p.i. of the radiotracer by normalization for body weight of the animal and injected dose. Since PDE10A is expressed at low level in the cerebellum, binding potential (BP) values were calculated using the multilinear reference tissue model developed by Ichise et al. with the cerebellum considered as reference, using the same software (PMOD). Rats (n=5) were used for baseline imaging of which 3 animals were used in pretreatment studies in which the non-radioactive analogue B-1 was administered subcutaneously at a dose of 5 mg/kg at 30 min prior to the radiotracer injection.

In accordance to the biodistribution study, [¹⁸F]B-1 was readily taken up in the brain, and peak tissue concentrations were reached within one minute. This was followed by a slow washout of the tracer from the striatum, which is known to have high expression of the PDE10A enzyme. Peak striatum-to-cerebellum ratios of 2.7 were obtained at 15 min post injection and the ratios remained to be higher than 2.5 until about 35 min post injection. After this, the ratios decreased slowly until the end of the experiments (90 min). In the images, high intensity signal was observed in the striatum with only background radioactivity in the cortical regions as well as in the cerebellum. When the animals were pre-treated with the non-radioactive analogue B-1, the striatum-to-cerebellum ratios decreased to 1.5. Also, there was about 80% reduction in BP values with pretreatment experiments compared to baseline scans. The mean BP value at baseline was 0.58±0.17 (n=5) whereas after administration of non-radioactive analogue, the value decreased to 0.11±0.10 (n=3).

Fluorine-18 labeled tracer was synthesized for imaging of PDE10A in vivo. Biodistribution studies as well as microPET imaging studies have shown specific retention or slower washout of this tracer from PDE10A rich region striatum. Therefore, [¹⁸F]B-1 is a suitable agent for imaging and quantification of PDE10A using PET.

### E. Studies with [¹⁸F]B-2

### E.1. Biodistribution Studies

The biodistribution study of [¹⁸F]B-2 was carried out in male Wistar rats (body weight 270-340 g) at 2 min, 30 min and 60 min post injection (p.i.) (n=3/time point). Rats were injected with about 1.1 MBq of the tracer via tail vein under anesthesia (2.5% Isoflurane in O₂ at 1 L/min flow rate) and sacrificed by decapitation at above specified time points. Blood and major organs were collected in tared tubes and weighed. The radioactivity in blood, organs and other body parts was measured using an automated gamma counter The distribution of radioactivity in different parts of the body at different time points p.i. of the tracer was calculated and expressed as percentage of injected dose (% ID), and as percentage of injected dose per gram tissue (% ID/g) for the selected organs. % ID is calculated as cpm in organ/total cpm recovered. For calculation of total radioactivity in blood, blood mass was assumed to be 7% of the body mass.

The results of the in vivo distribution study of [¹⁸F]B-2 in male Wistar rats is presented in Table 8 and 9. Table 8 shows the % injected dose (% ID) values at 2 min, 30 min and 60 min post injection (p.i.) of the radiotracer. At 2 min p.i. about 4.0% of the injected dose was present in the blood, and this cleared to 2.1% by 60 min after injection of the tracer. The total brain uptake of the tracer at 2 min p.i. was 0.56%, with 0.45% of the ID in the cerebrum and 0.1% in the cerebellum, suggesting a rather lower (initial) uptake of [¹⁸F]B-2 in the brain. At 2 min p.i. 0.029% of ID was present in the striatum, which has the highest expression of PDE10A enzyme, and where the radiotracer is expected to show binding. The % ID in the striatum at 60 min p.i. is higher compared to that at 30 min (0.052% vs 0.043%) whereas in other organs the % ID values were similar or slightly lower at those time points because of the clearance of the tracer from these regions. This indicates accumulation of [¹⁸F]B-2 in the striatum due to binding to the PDE10 enzyme in this region. At 60 min p.i. 2.1% of ID was still present in the blood, indicating rather poor clearance of this tracer from the blood circulation. The compound was cleared mainly by hepatobiliary system as there was in total of 55% ID present in the liver and intestines at 60 min after injection of the radiotracer.

TABLE 8
Biodistribution of [¹⁸F]B-2 in normal rats at 2, 30 and 60 min p.i.

| | % ID ^{a} | | |
|---|---|---|---|
| Organ | 2 min | 30 min | 60 min |
| Urine | 0.12 ± 0.0 | 0.21 ± 0.0 | 0.86 ± 0.2 |
| Kidneys | 6.91 ± 0.5 | 1.80 ± 0.1 | 1.35 ± 0.1 |
| Liver | 41.06 ± 2.4 | 52.32 ± 4.6 | 42.61 ± 2.3 |
| Spleen + Pancreas | 1.93 ± 0.1 | 0.62 ± 0.1 | 0.38 ± 0.0 |
| Lungs | 2.39 ± 0.1 | 0.37 ± 0.0 | 0.34 ± 0.0 |
| Heart | 0.93 ± 0.1 | 0.25 ± 0.0 | 0.21 ± 0.0 |
| Stomach | 1.69 ± 0.3 | 5.14 ± 2.0 | 13.70 ± 1.4 |
| Intestines | 9.39 ± 0.6 | 10.81 ± 4.9 | 12.53 ± 2.5 |
| Striatum | 0.029 ± 0.005 | 0.043 ± 0.010 | 0.052 ± 0.001 |
| Hippocampus | 0.011 ± 0.003 | 0.008 ± 0.000 | 0.008 ± 0.001 |
| Cortex | 0.072 ± 0.002 | 0.024 ± 0.004 | 0.021 ± 0.002 |
| Rest of cerebrum | 0.334 ± 0.010 | 0.191 ± 0.011 | 0.176 ± 0.005 |
| Cerebrum total | 0.447 ± 0.009 | 0.266 ± 0.003 | 0.257 ± 0.007 |
| Cerebellum | 0.101 ± 0.015 | 0.035 ± 0.003 | 0.031 ± 0.002 |
| Blood | 3.97 ± 0.3 | 2.05 ± 0.2 | 2.14 ± 0.2 |
| Carcass | 32.77 ± 2.4 | 26.36 ± 1.5 | 26.04 ± 1.3 |

Data are expressed as mean ± SD; n = 3 per time point;
^{a} Percentage of injected dose calculated as cpm in organ/total cpm recovered

Because of its lipophilic character, the urinary excretion of the tracer was minimal with only ˜2.2% ID present in the urinary system at 60 min p.i. In view of the large mass of the carcass, significant amount of the injected dose (˜28% ID) was present in the carcass at all time points examined. Typically, carcass constitutes to >90% of the total body weight of the animal Table 9 shows the % ID/g values for different organs at 2 min, 30 min and 60 min p.i.

TABLE 9
[¹⁸F]B-2 concentration in different organs at 2, 30 and 60 min p.i.

| | % ID/g ^{a} | | |
|---|---|---|---|
| Organ | 2 min | 30 min | 60 min |
| Kidneys | 2.50 ± 0.18 | 0.62 ± 0.06 | 0.47 ± 0.05 |
| Liver | 3.21 ± 0.27 | 4.25 ± 0.73 | 3.15 ± 0.10 |
| Spleen + Pancreas | 1.29 ± 0.05 | 0.36 ± 0.02 | 0.26 ± 0.03 |
| Lungs | 1.74 ± 0.33 | 0.28 ± 0.01 | 0.24 ± 0.03 |
| Heart | 0.95 ± 0.08 | 0.25 ± 0.02 | 0.21 ± 0.01 |
| Striatum | 0.56 ± 0.10 | 0.61 ± 0.03 | 0.81 ± 0.06 |
| Hippocampus | 0.27 ± 0.01 | 0.14 ± 0.01 | 0.12 ± 0.00 |
| Cortex | 0.43 ± 0.04 | 0.16 ± 0.01 | 0.14 ± 0.01 |
| Rest of cerebrum | 0.35 ± 0.01 | 0.18 ± 0.01 | 0.17 ± 0.01 |
| Cerebrum total | 0.36 ± 0.01 | 0.20 ± 0.01 | 0.20 ± 0.01 |
| Cerebellum | 0.42 ± 0.01 | 0.13 ± 0.00 | 0.11 ± 0.01 |
| Blood | 0.19 ± 0.06 | 0.10 ± 0.01 | 0.09 ± 0.01 |
| Cerebrum + | 0.38 ± 0.11 | 0.20 ± 0.00 | 0.19 ± 0.01 |
| Cerebellum | | | |

Data are expressed as mean ± SD; n = 3 per time point;
^{a} % ID/g values are calculated as % ID/weight of the organ in g.

As kidneys and liver are the excretory organs, they have the highest % ID/g values with about 2.5% ID/g for kidneys and 3.2% ID/g for liver at 2 min p.i. The % ID/g values for different regions of brain, namely striatum, hippocampus, cortex and cerebellum are presented in Table 9. At 2 min p.i. the radioactivity concentration in the striatum was about 0.56% ID/g. In the hippocampus, cortex and cerebellum, regions with minimal expression of the enzyme, the % ID/g was lower, respectively, 0.27% ID/g, 0.43% ID/g and 0.42% ID/g. At 60 min p.i., the radioactivity concentration in the hippocampus, cortical regions and the cerebellum decreased to <0.14% ID/g, while the intensity of the tracer in the striatum had significantly increased at 60 min p.i. compared to 30 min p.i. (0.81 vs 0.61% ID/g). This indicates an accumulation of [¹⁸F]B-2 in the striatum, whereas for other regions of the brain there is a clear washout. These findings are consistent with the higher expression of the PDE10 enzyme in the striatum. The % ID/g tissue values were normalized for their body weight in order to correct for differences in body weight between different animals. The normalized values are presented in Table 10.

TABLE 10
[¹⁸F]B-2 concentration in different organs at 2, 30
and 60 min p.i. normalized for the body weight of the animal

| | % ID/g × kg ^{a} | | |
|---|---|---|---|
| Organ | 2 min | 30 min | 60 min |
| Kidneys | 0.73 ± 0.08 | 0.17 ± 0.02 | 0.15 ± 0.01 |
| Liver | 0.94 ± 0.08 | 1.21 ± 0.22 | 1.02 ± 0.07 |
| Spleen + Pancreas | 0.38 ± 0.02 | 0.10 ± 0.00 | 0.08 ± 0.01 |
| Lungs | 0.51 ± 0.11 | 0.08 ± 0.00 | 0.08 ± 0.01 |
| Heart | 0.28 ± 0.02 | 0.07 ± 0.00 | 0.07 ± 0.00 |
| Striatum | 0.164 ± 0.04 | 0.172 ± 0.01 | 0.263 ± 0.02 |
| Hippocampus | 0.080 ± 0.00 | 0.041 ± 0.00 | 0.040 ± 0.00 |
| Cortex | 0.124 ± 0.01 | 0.045 ± 0.00 | 0.046 ± 0.00 |
| Rest of cerebrum | 0.101 ± 0.01 | 0.053 ± 0.00 | 0.056 ± 0.00 |
| Cerebrum total | 0.106 ± 0.01 | 0.058 ± 0.00 | 0.064 ± 0.00 |
| Cerebellum | 0.122 ± 0.01 | 0.037 ± 0.00 | 0.037 ± 0.00 |
| Blood | 0.06 ± 0.00 | 0.03 ± 0.00 | 0.03 ± 0.00 |
| Cerebrum + | 0.11 ± 0.01 | 0.06 ± 0.00 | 0.06 ± 0.00 |
| Cerebellum | | | |

Data are expressed as mean ± SD; n = 3 per time point;
^{a} % ID/g × kg values are calculated as % ID/weight of the organ in g multiplied by the body weight of the animal in kg.

TABLE 11
Clearance of [¹⁸F]B-2 from different regions of the brain calculated
as 2 min to 30 min or 2 min to 60 min ratio for % ID/g values

| Brain region | 2 min/30 min | 2 min/60 min |
|---|---|---|
| Striatum | 0.92 | 0.69 |
| Hippocampus | 1.90 | 2.21 |
| Cortex | 2.69 | 3.04 |
| Rest of cerebrum | 1.87 | 2.02 |
| Cerebrum total | 1.78 | 1.84 |
| Cerebellum | 3.17 | 3.68 |
| Blood | 1.88 | 2.05 |

Table 11 shows 2 min/30 min and 2 min/60 min ratios for % ID/g values for different regions of the brain. For the 2 min/60 min ratios, the cerebellum has the highest ratio of 3.68, indicating that the clearance of the radioactivity is the fastest from this region, followed by other regions of the brain such as cortical areas and the hippocampus, which do not express PDE10A. For the striatum, the 2 min/60 min ratio (0.69) is lower then the 2 min/30 min ratio (0.92) indicating accumulation of [¹⁸F]B-2 in this PDE10 rich region.

TABLE 12
Striatum-to-other brain region ratios (calculated from %
ID/g values) at 2 min, 30 min and 60 min p.i. of [¹⁸F]B-2

| Brain region | 2 min | 30 min | 60 min |
|---|---|---|---|
| striatum/hippocampus | 2.04 | 4.22 | 6.53 |
| striatum/cortex | 1.31 | 3.84 | 5.77 |
| striatum/cerebellum | 1.33 | 4.61 | 7.10 |
| striatum/blood | 2.88 | 5.90 | 8.56 |

Table 12 presents the ratios between striatum and other regions of the brain as well as blood at different time points post injection of [¹⁸F]B-2. Striatum was considered as a PDE10-rich region and cerebellum as a reference region. Therefore, high striatum to cerebellum ratio is desired in order to have good quality images in vivo. At 2 min p.i., the striatum to cerebellum ratio was about 1.33 and this ratio increased to 7.10 by 60 min after injection of the tracer. Striatum to cortex and striatum to hippocampus ratios were also ≧5.8, confirming the specific retention of [¹⁸F]B-2 in the striatum.

### E.2. Plasma Metabolite Analysis

The metabolic stability of [¹⁸F]B-2 was studied in normal rats by determination of the relative amounts of parent tracer and metabolites in the blood at 1 hour p.i. of the tracer. After intravenous (i.v.) administration of about 0.5 mCi [¹⁸F]B-2 via tail vein under anesthesia (2.5% Isoflurane in O₂ at 1 L/min flow rate), rats were sacrificed by decapitation at 60 min p.i. (n=2). Blood was collected in heparin containing tubes (4.5 mL LH PST tubes; BD vacutainer, BD, Franklin Lakes, N.J., USA) and immediately centrifuged for 10 min at 3000 rpm to separate the plasma. About 0.4 mL of this supernatant plasma was spiked with about 5 μg of non-radioactive B-2 (1 mg/mL solution) and injected on to HPLC, which was connected with a Chromolith performance column (C₁₈, 3 mm×100 mm, Merck KGaA, Darmstadt, Germany). The mobile phase consisted of 0.05 M NaOAc pH 5.5 (solution A) and acetonitrile (solvent B). The following method was used for the analysis: isocratic elution with 100% A for 4 min at a flow rate of 0.5 mL/min, then linear gradient to 90% B by 14 min at a flow rate of 1 mL/min, and isocratic elution with mixture of 10% A and 90% B until 17 min. After passing through UV detector (254 nm), the HPLC eluate was collected as 1-mL fractions using an automatic fraction collector and the radioactivity of these fractions was measured in the γ-counter.

The metabolic stability was assessed in plasma collected from normal rats (n=2) 60 min post injection of [¹⁸F]B-2 using RP-HPLC. The cold intact compound B-2 was co-injected onto HPLC to identify the intact parent tracer. An overview of the results from the plasma metabolite analysis is presented in Table 13.

TABLE 13
Relative percentages of intact tracer and metabolites
in rat plasma at 60 min p.i. of [¹⁸F]B-2

| (%) | mean ± SD (n = 2) |
|---|---|
| Polar metabolite(s) | 26.8 ± 3.7 |
| Intact tracer | 70.8 ± 5.4 |
| Apolar components | 2.4 ± 1.6 |

The analysis showed that at 60 min post injection of the radiotracer, about 71% of the recovered radioactivity was in the form of intact tracer and about 27% of the activity was in the form of polar metabolites. The radioactivity corresponding to the (lipophilic) fractions eluting after the intact tracer was negligible (˜2%).

### E.3. Brain Metabolite Analysis

From the same animals that were used for plasma analysis, brain was also dissected and rinsed with saline (Mini Plasco®, Braun, Melsungen, Germany) to rinse off the blood. Cerebrum and cerebellum were collected separately and homogenized in 3 mL and 2 mL of acetonitrile, respectively, for about 3 min. A volume of 1 mL of this homogenate was diluted with an equal volume of water and a part of this homogenate was filtered through 0.22 μm filter (Millex®-GV, Millipore, Ireland). About 0.5 mL of the filtrate was spiked with 5 μL of non-radioactive B-2 solution (1 mg/mL) and then injected on to HPLC, which was connected with an XBridge™ column (C₁₈, 5 μM, 3 mm×100 mm, Waters) eluted with mixtures of 0.05 M sodium acetate buffer pH 5.5 and acetonitrile (65:35 v/v) at a flow rate of 0.8 mL/min. Similar to the plasma analysis, the HPLC eluate was collected as 1-mL fractions after passing through the UV detector, and the radioactivity in the fractions was measured using the automated gamma counter.

The above plasma analysis indicates that [¹⁸F]B-2 is metabolized to more polar radiometabolites. Based on their higher hydrophilicity we can expect that these radiometabolites may not cross BBB. However, in order to rule out the presence or entry of these metabolites to the brain, we have also analyzed cerebrum and cerebellum samples of rats after isolation and subsequent homogenization at 60 min after injection of [¹⁸F]B-2 (n=2).

TABLE 14
Relative percentages of intact tracer and metabolites in
rat cerebrum and cerebellum at 60 min p.i. of [¹⁸F]B-2.

| (%) | Cerebrum | Cerebellum |
|---|---|---|
| Polar metabolite(s) | 18.5 ± 0.2 | 31.9 ± 3.2 |
| Intact tracer | 81.0 ± 0.4 | 66.8 ± 4.5 |
| Apolar metabolite(s) | 0.4 ± 0.2 | 1.3 ± 1.3 |

Results are presented as mean ± SD (n = 2)

Table 14 gives the overview of the results of the radiometabolite assay in the cerebrum as well as cerebellum at 60 min after injection of [¹⁸F]B-2. About 81% of the recovered radioactivity from the cerebrum homogenate analysis was in the form of the parent tracer [¹⁸F]B-2. In the cerebellum tissue about 67% of the recovered radioactivity was present as the intact tracer. Polar metabolites (18.5% in cerebrum and 32% in cerebellum) were detected in brain. However, since the rat was not perfused, the presence of polar metabolites could be (partly) due to the blood that was still present in brain. The percentage of lipophilic metabolites in the brain was negligible, 0.4% in cerebrum and 1.3% in cerebellum.

### E.3. Perfused Brain Radiometabolite Analysis

For each studied time point two rats were injected with about 37 MBq of [¹⁸F]B-2. At 30 min or 60 min p.i., the rats were sacrificed by administering an overdose of Nembutal (CEVA Sante Animale, 200 mg/kg intraperitoneal). The rats were perfused by injection of saline into the right ventricle until the liver turned pale. Brain was isolated, cerebrum and cerebellum were separated and homogenized in 3 mL and 2 mL of acetonitrile respectively, for about 3 min. A volume of 1 mL of this homogenate was diluted with an equal volume of water and 1 mL of the supernatant was filtered through a 0.22 μm filter (Millipore, Bedford, USA). About 0.5 mL of the filtrate was diluted with 0.1 mL of water and spiked with 5 μL of non-radioactive B-2 solution (1 mg/mL). A volume of 0.5 mL of the homogenate extracts were injected onto an HPLC system consisting of an analytical XBridge column (C₁₈, 5 μM, 3 mm×100 mm, Waters) eluted with a mixture of 0.05 M sodium acetate (pH 5.5) and CH₃CN (65:35 v/v) at a flow rate of 0.8 mL/min. The HPLC eluate was collected as 1-mL fractions after passing through the UV detector (254 nm), and radioactivity in the fractions was measured using an automated gamma counter.

An overview of the results from the perfused rat brain radiometabolite analysis is presented in Table 15.

TABLE 15
Relative percentages of intact tracer and radiometabolites in perfused
rat cerebrum and cerebellum at 30 and 60 min p.i. of [¹⁸F]B-2.

| | 30 min p.i. | | 60 min p.i. | |
|---|---|---|---|---|
| (%) | Cerebrum | Cerebellum | Cerebrum | Cerebellum |
| polar metabolite | 4.8 ± 2.0 | 9.7 ± 3.5 | 14.4 ± 0.6 | 25.8 ± 4.3 |
| intact tracer | 95.2 ± 2.0 | 90.3 ± 3.5 | 85.6 ± 0.6 | 74.2 ± 4.2 |

Results are presented as mean ± SD (n = 2)

The fraction of polar radiometabolites detected in cerebellum is higher compared to cerebrum. At 30 min p.i. about 95% of the recovered radioactivity was present as intact tracer in cerebrum, in cerebellum this was ˜90%. After 60 min, the amount of intact tracer in cerebrum decreased to ˜86%, in cerebellum this was about 74%.

E.4. microPET Imaging Study

Imaging experiments were performed on a Focus™ 220 microPET scanner (Concorde Microsystems, Knoxville, Tenn., USA) using male Wistar rats with body weight between 350 and 480 g. For the baseline microPET study, rats (n=2) were injected with about 74 MBq of high specific activity formulation of [¹⁸F]B-2 via tail vein under anesthesia (2.5% Isoflurane in O₂ at 1 L/min flow rate). For the pretreatment experiment, the radioactivity dose was reduced to about 37 MBq. Pretreatment was done by administering the non-radioactive analogue B-2 subcutaneously at a dose of 2.5 mg/kg at 60 min prior to the tracer injection (n=1).

Dynamic scans of 120 min were acquired in List mode. Acquisition data were Fourier rebinned in different time frames (4×15 s, 4×1 min, 5×3 min, 8×5 min, 10×6 min) and reconstructed with Filtered Back Projection (FBP). An average image (frame 1 to 31) of the reconstructed data was spatially normalized to an in-house created [¹¹C]raclopride template of the rat brain in Paxinos coordinates. The affine transformation was then used to normalize all time frames of the dynamic dataset to allow automated and symmetric volumes of interest (VOIs) analyses. Time-activity curves (TAC) were generated for striatum, cortex and cerebellum for each individual scan, using PMOD software (v 3.0, PMOD Technologies Ltd., Zurich, Switzerland). The radioactivity concentration in the different brain regions was expressed as standardized uptake value (SUV) as a function of time p.i. of the radiotracer by normalization for body weight of the animal and injected dose. Since PDE10A is expressed at low level in the cerebellum, binding potential (BP) values were calculated using the multilinear reference tissue model developed by Ichise et al. with the cerebellum considered as reference, using the same software (PMOD).

After injection of [¹⁸F]B-2, there is a high initial uptake of the radiotracer in the striatum, cortical regions and the cerebellum. After this initial high uptake due to the blood pool activity, the radioactivity cleared from the cortical regions and the cerebellum, however it was still increasing in the striatum where the PDE10 enzyme is expressed in the highest concentration. In the images, high intensity signal was observed in the striatum with only background radioactivity in the cortical regions as well as in the cerebellum. At baseline a maximum average striatum-to-cerebellum ratio of 3.9 was reached after about 32 min p.i., which stayed constant until about 75 min p.i. Self-blocking at dose 2.5 mg/kg resulted in a strong reduction of the BP value from 3.3 at baseline to equal or less than 1.4 after pretreatment.

Fluorine-18 labeled tracer was synthesized for imaging of PDE10A in vivo. Biodistribution studies as well as microPET imaging studies have shown specific retention of this tracer in PDE10A rich region striatum. Therefore, [¹⁸F]B-2 is a suitable agent for imaging and quantification of PDE10A using PET.

1. A compound of Formula (I) 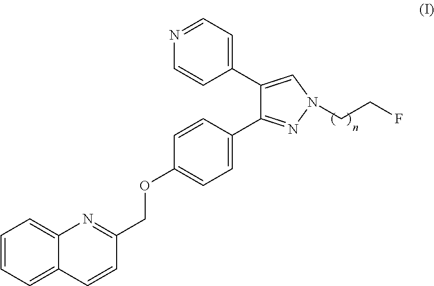
wherein n is 1 or 2, and F is [¹⁸F], or an acceptable salt or a solvate thereof.

2. The compound according to claim 1 wherein n=1.

3. The compound according to claim 1 wherein n=2.

4. A sterile composition comprising a compound of formula (I) of claim 1 dissolved in saline.

5. (canceled)

6. A method of imaging a tissue, cells or a host, comprising contacting with or administering to a tissue, cells or a host a compound of claim 1, and imaging the tissue, cells or host with a PET imaging system.
